(19) United States
(12) Patent Application Publication
(10) Pub. No.: US 2014/0050801 A1
(43) Pub. Date: Feb. 20, 2014

(54) GAS DISSOLVING APPARATUS

(71) Applicant: Caius Araujo Martins de Camargo, Sao Paulo - SP (BR)

(72) Inventor: Caius Araujo Martins de Camargo, Sao Paulo - SP (BR)

(73) Assignee: G TECH LICENSING, LLC, Grosse Pointe, MI (US)

(21) Appl. No.: 14/114,162

(22) PCT Filed: Apr. 26, 2011
(86) PCT No.: PCT/US11/33935

§ 371 Date: Oct. 25, 2013

(51) Int. Cl. B01F 3/04; A61K 33/00; A01N 59/00; A61K 33/14

(52) U.S. Cl. CPC B01F 3/04985; A61K 33/14; A61K 33/00; A01N 59/00
USPC 424/615; 366/336; 210/620; 426/7; 426/397; 424/613

(57) ABSTRACT

A gas dissolving apparatus that combines a gas at first pressure into a working fluid, the working present at a second pressure equal to or greater than the first pressure. The device includes a molecular mixing chamber which is designed as a truncated conical chamber located between an inlet

### BACKGROUND

The present invention is directed to an apparatus which supplies dissolved gases (such as oxygen, ozone, chlorine, etc.) for chemical and biological processes. There are several industrial applications for gas dissolving in liquid fluids, but above all of them, the key drivers are environmental responsibility and health care.

Suggested preliminary classification in one or more of the following: C02F 1/24; C02F 1/40; C02F 1/72; C02F 3/02; B01F 1/00; B03D; A01K 63/04; A61L.

Over the years, and based on the international community consensus, the United Nations has complied universal understanding on human rights, labor standards, environmental preservation and anti-corruption. The work resulted in ten main principles which need to be embraced, supported and enacted as core values for organizations worldwide.

Principles 7, 8 and 9 pertain to the environment and they are: Principle 7: business should support a precautionary approach to environmental challenges; Principle 8: undertake initiatives to promote greater environmental responsibility; and Principle 9: encourage the development and diffusion of environmentally friendly technologies. It is highly desirable to provide processes and apparatus that align with the international community highest standards and core values of the most import matters for life as recognized by the United Nations.

From the foregoing, it can be appreciated that there exists a long felt need for methods and devices that can be utilized to produce oxygenated liquids in an effective and efficient manner. It is also posited that there exists a need and desire to produce fluid materials that are composed of mixtures of other gaseous and/or liquid compounds. Finally, it is posited that there exists a need for fluids having unique enrichment characteristics and that such materials have been difficult, if not impossible to obtain.

### SUMMARY

A gas dissolving apparatus that combines a gas present at a first pressure into a working fluid present at a second pressure that is equal to or greater than the first pressure. The device includes a molecular mixing chamber which is designed as a truncated conical chamber located between an inlet and an outlet. The device can include a plurality of inlets for the gas to enter into the mixing section, and a plurality of passages through the truncated conical chamber. The truncated conical chamber is surrounded by a cylindrical chamber leading to the outlet of the chamber.

### BRIEF DESCRIPTION OF THE DRAWINGS

The various features, advantages and other uses of the present apparatus will become more apparent by referring to the following detailed description and drawing in which:

FIG. 1 depicts a schematic side view of an embodiment of gas dissolving apparatus and its components as disclosed herein;

FIG. 2 is a perspective view of an embodiment of the gas dissolving apparatus functioning in an aerator for wastewater treatment process apparatus;

FIG. 3 is a perspective view of an embodiment of the gas dissolving apparatus functioning in a disinfection device for swimming pool water; and

FIG. 4 is a perspective view of an embodiment of the gas dissolving apparatus functioning in a cluster of aerators for enhancing oxygen dissolved levels in rivers and other depleted large bodies of water.

### DETAILED DESCRIPTION

Disclosed herein is an apparatus for integrating gas into a fluid that includes a first tube configured to convey a stream of liquid from a first point to a second point; a helicoidally shaped element affixed inside the first tube; a constriction section located downstream of the first tube; a truncated cone having a first section wider than a second section with the wider section connected to the first tube and the second narrower section is connected to a second tube. The truncated cone defining an inner section. The apparatus also includes an outer chamber configured with at least one gas passage unit. The outer chamber containing the truncated cone, at least a portion of the second tube and at least a portion of the first tube. The inner chamber includes perforations defined in the truncated cone.

As broadly construed, the apparatus 10 includes a first tube (1) through which a stream of liquid is able to pass. The stream is generated by a regular pumping system connected to the said first tube (1). In any suitable manner the first tube (1) has a helicoidally shaped element (8) fixed inside. Helicoidally shaped element (8) is configured to increase spinning in the liquid stream as it passes through the helicoidally shaped element (8).

The liquid continues downstream the first tube to a constriction section (2), whereby it undergoes a considerable constriction. This results in an increase in the velocity of the stream. The relationship between the tube diameter and the size of the constrictor opening is such that the velocity increase can be at a ratio of between 1:2 to 1:10 or greater.

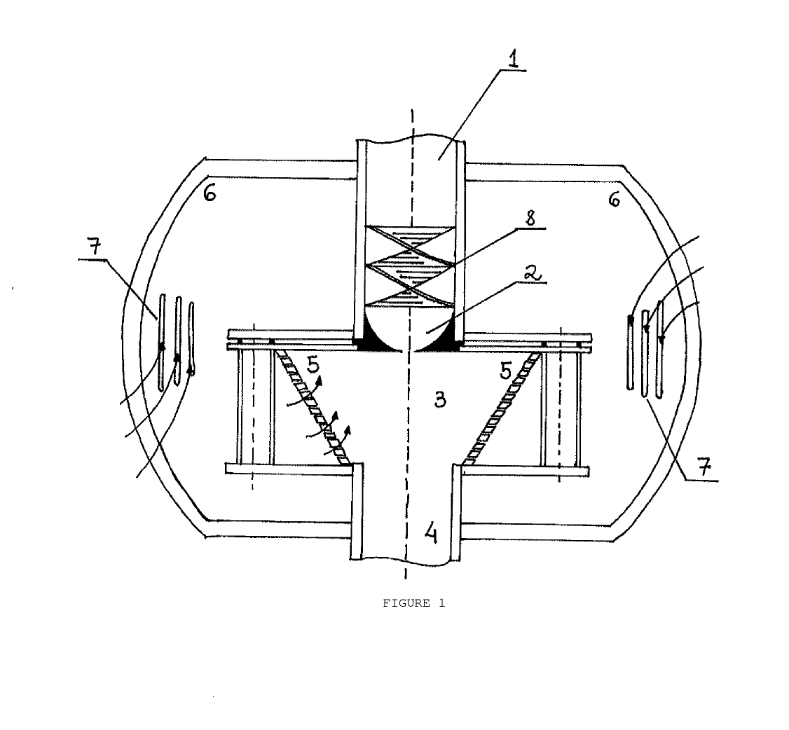

In the embodiment depicted in FIG. 1, the constrictor is positioned proximate the exit end of the first tube (1).

The constriction section (2) can have a suitable constrictor configuration. One non-limiting example of a constriction section (2) is depicted in the drawing figures. The constriction section has a lateral wall having a tapered or curvilinear face that is oriented toward the oncoming fluid flow and an opposed face. The tapered or curvilinear face tapers from a maxima located near the outer perimeter of the lateral wall and a narrowed section proximate to a central opening. The central opening has any suitable geometry such as circular, ovoid or the like. The area of the central opening will be less than the cross sectional area of the first tube. The ratio between the respective cross sectional areas will be suitable to produce increased fluid stream velocity as it traverses the central opening.

The constriction section (2) opens to inner chamber (3). Inner chamber (3) is defined by a walled body having an upstream region and a downstream, region. The upstream region of the walled body has a cross sectional diameter greater that the central opening in the constriction section (2). In many embodiments, the diameter of the upstream region of the chamber will be greater than the cross sectional diameter of the first tube (1).

In the embodiment depicted in the drawing figures, the inner chamber comprises a truncated cone with the wider radius region connected to the first tube (1) and the narrow radius connected to a second tube (4). The truncated cone is configured to convey the fluid stream from the central opening defined in the constriction section to the opening defined in the first end of second tube (4).

Second tube (4) can function as an exit from inner chamber (3). The truncated cone can be connected to the first tube (1) by any suitable means. Non-limiting examples include welding, soldering integral molding, etc. In the embodiment set forth in FIG. 1 the wider section of the truncated cone engages a face plate member. An aperture is defined in the central portion of the face plate member. The outer surface of the first tube (1) engages the face plate member. In the embodiment depicted the face plate member is composed of two planar members with one engaging the outer surface of the first tube (2) and one engaging an outer surface of the constriction member.

As the liquid stream passes through the inner chamber (3), it faces and abrupt enlargement from the constricted section (2) it was exiting in to the inner chamber. The velocity of the fluid stream may experience reduction as it traverses the inner chamber. The enlargement experienced as the fluid stream enters the inner chamber (3) results in the creation of a pressure reduction generating depression and a suction effect inside the inner chamber (3). The suction effect is sufficient to draw gas into the inner chamber (3) and to create an environment in which the gas resident in the inner chamber and the liquid transiting the inner chamber are facing extreme turbulence, facing different pressures and mixing together to create a new mass volume. The fluid flow in the inner chamber (3) has attributes of a vortex.

The inner chamber (3) wall(s) can be configured with suitable apertures or channels to facilitate the inflow of gas to compensate for the pressure reduction. The size and number of openings defined in the inner chamber wall are dependent, at least in part, upon parameters such as fluid flow through the device and the desired concentration of gas to be introduced into the fluid. Where the inner chamber is configured with a truncated cone, the conical walls can have a plurality of perforations distributed thereon. The density to the perforations will be that sufficient to permit gas transfer but will be less than that amount that would compromise wall structure. Other factors that may affect the density and/or number of the perforations employed can include the viscosity of the liquid being transferred through the apparatus. For example, for fluids have densities at or near the density of water, higher perforation density is desirable in certain situations. It is contemplated that that perforation density may vary depending on the density of the fluid conveyed and treated.

The inner chamber (3) is surrounded by a larger outer chamber (6). Outer chamber (6) is filled with the gas needed to be dissolved. The outer chamber is configured with inlets (7) to let the gas enter and fill its space. The wall (5) of the inner chamber (3) has many small perforations in order to let the passage of the gas from the outer chamber (6) to the inner chamber (3), whereby the suction effect present in the inner chamber (3), as previously described, draws the gas from the outer chamber (6).

Inside the inner chamber (3), the liquid stream meets the gas and creates a strong turbulence, providing an environment conducive for dissolving the gas into the liquid. The new liquid stream combined with the gas passes through the exit tube (4) on to suitable uses. The exit tube (4) has a diameter narrower than or equal to the first tube (1). This creates an increase in pressure providing an environment conducive for increasing the rate of gas dissolved in the liquid. The resulting fluid with elevated levels of dissolved gas can be employed in many useful applications.

It should also be noted that the suction effect produced by the process taking place in the apparatus can draw more gas into its chambers than is possible to be dissolved. Given turbulent flow and suction the apparatus as disclosed herein can trigger the production of micro bubbles that generate in the fluid stream upon the exit from the apparatus. These bubbles can be formed in thousands of sizes. Such fluid may have a variety of end-use applications such as use for flotation purposes.

The gas dissolving apparatus disclosed herein was conceived using sustainable engineering concepts, the efficient utilization of natural resources as its core value. Without being bound to any theory, it is believed that the apparatus as disclosed facilitates oxygenation. It is believed that the use of atmospheric gas or air results in dissolution of the air yielding efficiency levels thought to be achievable by other oxygenation processes only with the use of pure oxygen (85% plus oxygen concentration in the gas). Therefore the apparatus can significantly reduce costs, as its users are able to utilize free regular air to achieve desired results, instead of other costly methods and technologies. By combining free atmospheric air with the energy efficiency explained previously, the device disclosed herein is able to achieve a low long-term operation cost.

It is believed that the oxygenation potential can be utilized to treat bodies of water such as lakes, rivers, swimming pools etc. Other smaller capacity applications are also considered within the purview of this disclosure. The specific application for the apparatus will affect parameters such as apparatus through put and the like.

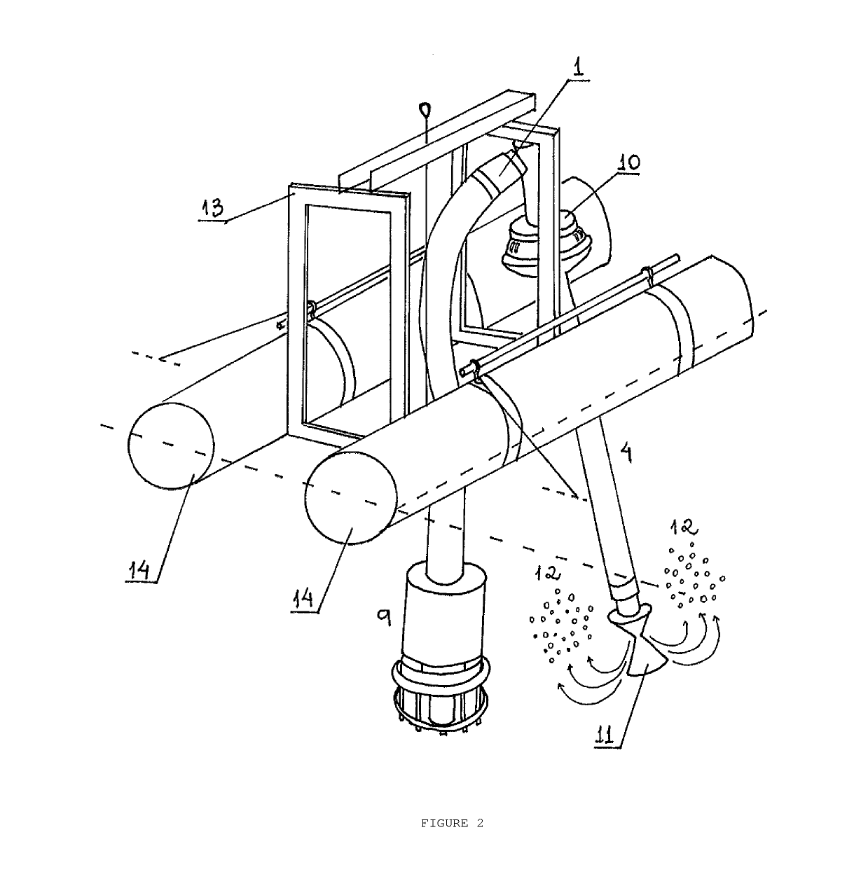

The apparatus disclosed herein can be utilized in several devices and applications. Some non-limiting examples of such applications are presented in the following discussion. In a first embodiment of a device utilizing the gas dissolving apparatus as disclosed herein, a green and sustainable solution is created using the invention as a component in an energy efficient and chemical free aerator as depicted in FIG. 2 which will be described in greater detail subsequently.

The gas dissolving apparatus as disclosed herein is energy efficient. The core technology was developed to dissolve high amounts of gas in fluids such as liquids while consuming less electricity or other energy than conventional equipment. The high level of efficiency is possible because the only source of energy utilized apparatus as disclosed comes from pumping systems that generate the initial stream of liquid. From that point onward, the apparatus does not require any complementary source of energy to accomplish gas dissolution.

The device disclosed is not only energy efficient, as mentioned, but as the liquid fluid mixes with gases inside the inner chamber it becomes instantly enriched with the gas, accelerating the dissolving process speed.

As mentioned previously, the gas dissolving apparatus as disclosed herein can be employed to promote flotation. Because the apparatus produces micro bubbles as a byproduct of the entire process, the resulting fluid can be used for flotation purposes. This effect happens because the apparatus draws in more gas than the amount possible to be dissolved into the liquid, resulting in the formation of micro bubbles on the exit tube. These bubbles are formed in thousands of sizes. The micro bubbles can be used to associate with target materials present in either the liquid stream or in larger bodies to which the liquid stream is introduced and raise the target materials to the surface where they can be skimmed or otherwise separated .of liquid. Non-limiting examples of such materials include oils, fats, biological waste, grease and suspended solids which may be present in the liquid and can raise to the surface upon exit promoting an efficient flotation effect for removal or separation. Thus the present disclosure comprehends a method for removing target materials from a liquid utilizing the device disclosed herein.

The present disclosure also contemplates a method of dissolving gas in liquids at elevated temperature utilizing the apparatus disclosed herein. According to the Henry' s Law of solubility, as the temperature of a liquid increases, any entrained gas becomes less soluble. Therefore, in warm liquids the gas dissolving process is a challenging task. The device as presently disclosed overcomes some for the main issues of warm liquids as the mixture of both gas and liquid occur at different pressures making it able to efficiently dissolve gas in liquids above 40 degrees Celsius.

The gas dissolving apparatus can be easily assembled. The device disclosed does not utilize any moving components. In the gas dissolving process utilizing the device disclosed herein, the liquid stream is created by means for generating a fluid steam such as a regular pumping system. The gas dissolving device disclosed herein can operate without the use of any moving component(s). This decreases overall maintenance cost and is significantly easier to assemble within a large-scale production line. Additionally, the gas dissolving apparatus represents long-term low cost operation in many situations. One contemplated application for the device disclosed herein is dissolving oxygen in liquid fluids. The oxygen dissolution can be accomplished using regular atmospheric air, which contains an average of 20% of oxygen.

The present disclosure also contemplates the inclusion of the gas dissolving apparatus into various assemblies having a moving fluid stream. One non-limiting example of such a device is in devices used in waste water treatment applications. For example, the gas dissolving apparatus can be utilized as an aerator for wastewater treatment processes. In one non-limiting examples as shown in FIG. 2, a submersible pump (9) creates a liquid stream composed of wastewater which runs through the tubing system (2) connected to the gas dissolving apparatus (10). Inside the apparatus the oxygen derived from atmospheric air is dissolved in the liquid stream and the resulting material travels downstream to the exit tube (4). In the embodiment depicted, exit tube (4) is vertically oriented and fixed relative to the device. The mixture (gas and liquid) travel through the exit tube (4) until reaching a diffuser (11) configured to disseminate the oxygen-enriched liquid in the associated wastewater treatment vessel.

It can be appreciated that the device depicted in FIG. 2 can be used to increase available oxygen the associated vessel thereby combating problems such as oxygen depletion. It is also contemplated that the device depicted in FIG. 2 can be employed to accomplish flotation of target materials such as entrained solid particles in wastewater by means of micro bubbles (12) present in the liquid stream upon the exit of the apparatus. Since atmospheric air is not completely dissolved while passing through the apparatus, it generates micro bubble in thousands of different sizes. These bubbles are responsible for lifting the suspended particles present in the wastewater for separation by processes such as skimming and the like.

The device depicted in FIG. 2 also presents a non-limiting example of the general format of an aerator as disclosed herein. The aerator includes flotation means such as a catamaran floating device using a stainless rigid framework (13) to connect the twin hulls (14), the tubing system (1), the gas dissolving apparatus core (10) and the submersible pumping system (9). As can be seen in this configuration, there was only one energy source for all the process; that used by the pumping system. The present disclosure contemplates that aeration can be accomplished using no additional, chemical additives (liquid, solid or gas) as the device uses free atmospheric air as the gas. However if desired or required, the aeration device as disclosed herein can be used in tandem with other water treatment devices and systems.

The present disclosure also contemplates as device for use with water recirculation as treatment systems such as might be employed in swimming pools and the like. In the second example of an apparatus, one non-limiting example of a health and entertainment solution is created, namely a disinfection process to be used in swimming pools.

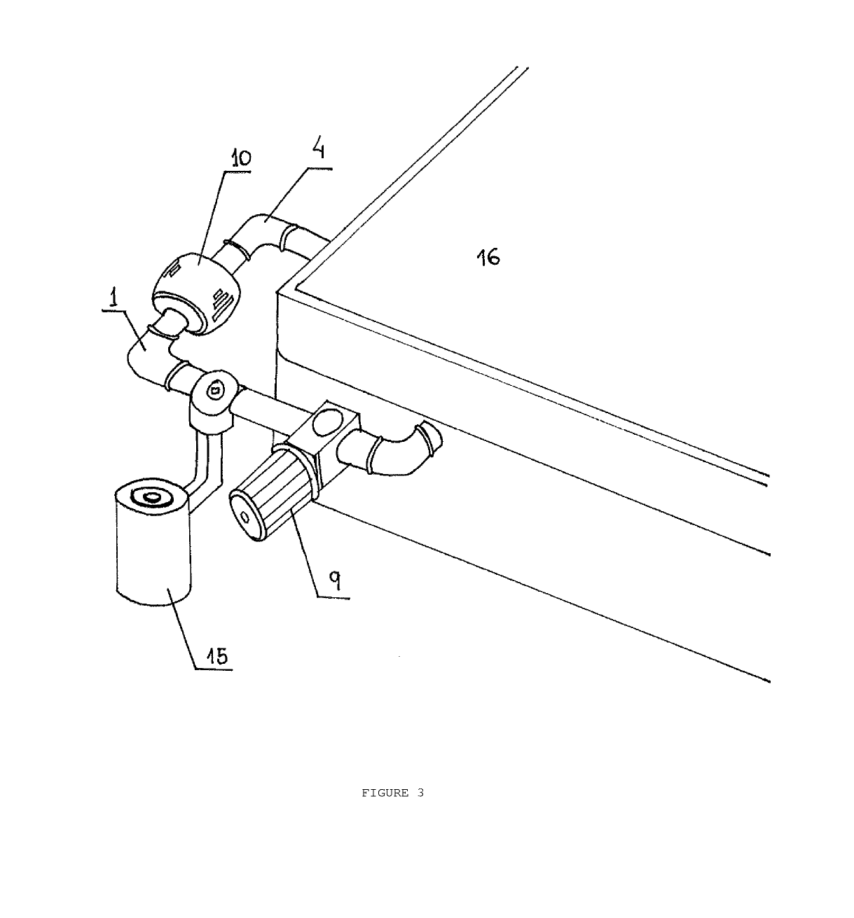

FIG. 3 is a perspective view of the second example of a device utilizing the gas dissolving apparatus in which the apparatus functions as a treatment and disinfection device for swimming pool water. As shown in FIG. 3, a pumping mechanism (9) is used to pump the water from the swimming pool (16) running it through a filter (15), such as those employed in most pool filtering system. After the water stream passes the filter (15), it runs through a tubing system (1) connected to the gas dissolving apparatus core (10). Inside the apparatus (10) the liquid stream dissolves the oxygen present in atmospheric air, which runs downstream to the exit tube (4) connected to the swimming pool (16), returning its oxygen-enriched filtered water. The disinfection process comprises subjecting microbial life to a liquid comprising of an oxygen-enriched liquid and also enriching the liquid containing microbial life with oxygen. The final effect of these double disinfection processes is reducing the chlorine used in the water and sometimes even avoiding its use completely.

In a third exemplary application of the gas dissolving apparatus, another green and sustainable solution is created using the invention: an energy efficient and chemical free aerator for atrophying rivers and other oxygen depleted large bodies of water.

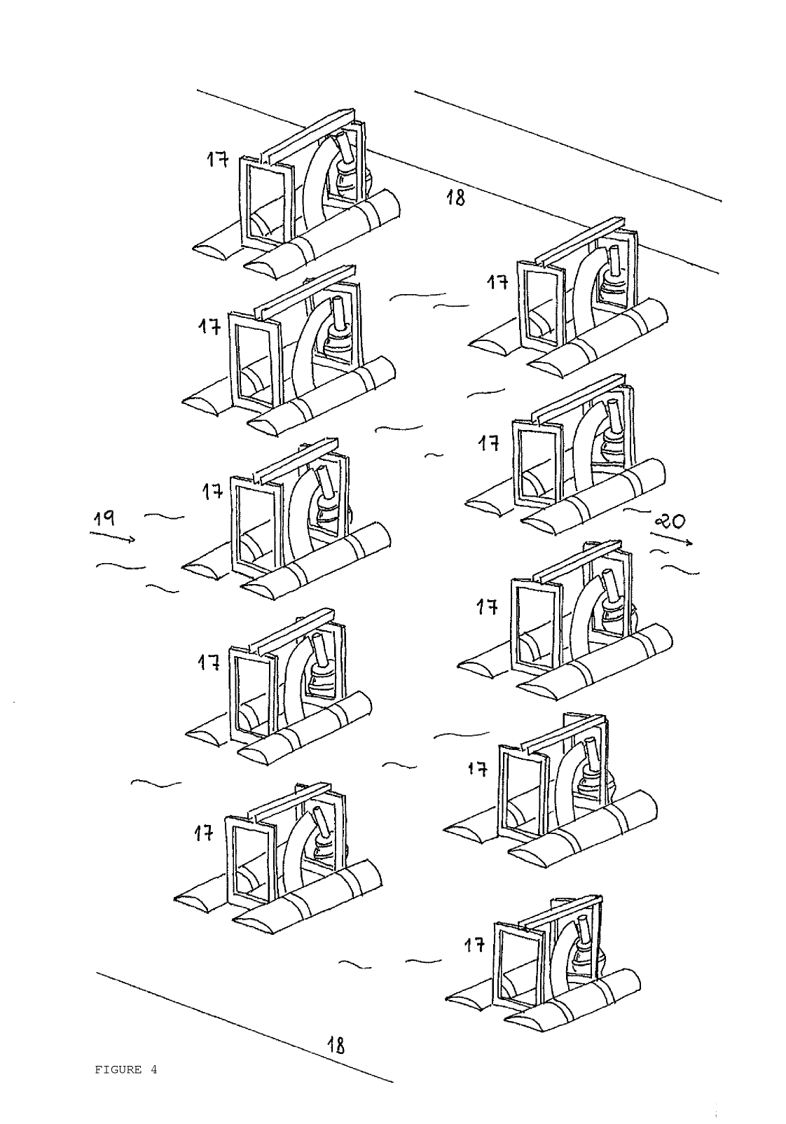

As depicted in FIG. 4, the gas dissolving apparatus is a component of a device functioning working as an aerator for depleted large bodies of water. As shown in FIG. 4, a number of floating aerators (17) similar to the ones described in conjunction with FIG. 2 are clustered together forming an island or treatment zone extending across the width between both of the banks (18) of the river. The oxygen-depleted water upstream the river (19) runs thought the cluster of aerators and is infused with water rich in dissolved oxygen from aerators (17) that enriches the dissolved oxygen level in the water downstream in the river (20). The oxygen-rich water enhancing the chances for supporting aquatic life in the downstream locations (20). The treatment also helps to disinfect the water by killing fecal coliform bacteria as well as reducing biological oxygen demand (BOD) and/or controlling odor, thereby bringing back enhanced water quality for the river.

Basically, when combined with pure oxygen or oxygen rich gases (e.g. atmospheric air), the invention promotes oxidation in liquid fluids—such as wastewater and industrial effluents.

The entire process results not only in the water treatment itself, but in particular cases, can also be part of the water recycling/reuse process.

Among its several applications on the environmental preservation and rehabilitation arena, the present invention is particularly suitable for processes including but not limited to 1) biodegradation of organic matter (such as in municipal and industrial wastewater treatment); 2) oxidation and precipitation of dissolved contaminants (e.g., iron, and manganese ions); 3) oxidation and destruction of dissolved organic contaminants in wastewater; 4) farming of aquatic species (such as fish and shrimp); 5) control of odors (such as those caused by anaerobic bacteria in contaminated wastewater or sludge); 6) killing of hazardous bacteria (e.g., Coliform bacteria); 7) bioremediation of contaminated (e.g., with petroleum products) or oxygen-depleted bodies of water; 8) rehabilitation of atrophying lakes; 9) biological oxygen demand (BOD) reduction techniques; 10) hydroponic agriculture; and/or 11) removal of pesticides in potable water, and in water to be discharged into public streams.

As for health care, it is also know that oxygenated liquids have therapeutic effects. For example, consumption of oxygen enriched beverages can have a favorable effect on well-being and physical performance, for it provides oxygen to the bloodstream through the stomach lining or intestinal wall. After a short period after ingestion of enriched water, there are evidences of a pulmonary function bypass as observed through an average blood oxygen level increase, and the effect of a concomitant cardiac relief was observed through an average pulse rate reduction.

There are more therapeutic processes in which an oxygenated liquid can be advantageously employed including, for example, oxygenation of wounds to increase the rate of healing and to reduce infections; oxygenated organ transplant storage media; tumor oxygenation for radiation therapy and chemotherapy; lung bypass by oxygenated liquids in case of pulmonary deficiencies; treatment for carbon monoxide poisoning; mouthwashes, dentifrices; topical, including cosmetic treatment media; contact lens treating solutions; and cell level therapeutic applications.

Oxygenated liquids may also be advantageously employed in some disinfection process. Such disinfection processes are those in which a very high level of dissolved oxygen is utilized to kill microbial life—as chlorine or ozone does. These oxygen concentration levels would exceed those resulting after dilution in a biomass for aerobic treatment thereof as described above. For example, it was found that a bacterium in a Petri dish was killed when merely subjected to oxygen-enriched water. It has also previously been speculated that rather than subjecting certain microbial life to a disinfectant comprising an oxygenated liquid, a disinfection process may instead involve oxygenating a liquid contaminated with microbial life, whereby the disinfection would take place during the oxygenation process. When used with waste water, the disinfection process ensures an even better water quality, as the killing of Coliform bacteria is one of the objectives of the water treatments.

It is also known that some fermentation processes, i.e., processes which involve fermenting a fermentation liquor, commonly employed in drug production or food processing by microorganisms, benefit from the fermentation liquor being comprised of an oxygenated liquid.

While the foregoing disclosure has been presented with respect to oxygenation of fluid streams, it is to be understood that the invention disclosed herewith can be employed to introduce various other gases including but not limited to chlorine, various halogens, nitrogen, helium and the like.

While the invention has been described in connection with what is presently considered to be the most practical and preferred embodiment, it is to be understood that the invention is not to be limited to the disclosed embodiments but, on the contrary, is intended to cover various modifications and equivalent arrangements included within the spirit and scope of the appended claims, which scope is to be accorded the broadest interpretation so as to encompass all such modifications and equivalent structures as is permitted under the law.

What is claimed is:

1. A gas dissolving apparatus comprising:
a first tube, the first tube having a first end and a second end downstream of the first end;
a turbulence-inducing member positioned in the first tube intermediate between the first and second ends;
a constrictor located proximate to the second end of the first tube downstream of the turbulence-inducing member;
an inner chamber element in fluid communication with the second end of the first tube, the inner chamber element having a truncated conical tapering wall member having a first upstream end and a second downstream end, wherein the first upstream end has a wider diameter than the second downstream end and wherein the first end of the truncated conical wall member is larger than the second end of the first tube, the truncated conical tapering wall member configured to facilitate transfer of gaseous material from a first region exterior to the first chamber to a second region located in the interior of the chamber element;
a second tube located downstream of the inner chamber, in fluid communication therewith, the second tube having a first and a second end downstream of the first end, wherein the first end of the second tube has a width equal to or less than the width of the first tube; and
an outer chamber element composed of an outer wall member having at least one gas transfer inlet defined therein, wherein the inner chamber element is contained within the outer chamber.

2. The gas dissolving apparatus of claim 1 wherein the turbulence inducing member has at least one region configured to induce spinning movement in a fluid stream passing through the first tube, the region having at least one helicoidal feature defined therein.

3. The gas dissolving apparatus of claim 2 wherein the turbulence inducing member is a heliciodal member.

4. The gas dissolving apparatus of claim 1 wherein the constrictor is configured to increase velocity in a liquid stream passing from the first tube into the inner chamber.

5. The gas dissolving apparatus of claim 1 wherein the truncated conical tapers wall has a plurality of perforations configured to permit the transfer of gas from a region exterior to the chamber.

6. The device of claim 1 wherein the device is employed is configured to process at least one of biomaterials, phenolics in oil.

7. A method for introducing gas into the a liquid stream in a dissolved state, the method comprising introducing liquid having a first gas concentration into the device defined of claim 1 at a first velocity and pressure, allowing the liquid stream to transit the device, the liquid stream exiting at a second velocity and pressure and elevated gas concentration, wherein the gas is air.

8. The method of claim 7 wherein the liquid is water.

9. A water recycling and reuse process comprising the steps of:
introducing waste water into the device of claim 1, the waste water having a first oxygen concentration upon introduction;
permitting the waste water to pass through the constrictor into the first chamber element of the device of claim 1, the passage into the inner chamber generating depression and a suction effect inside the inner chamber drawing gaseous oxygen into the inner chamber and the liquid stream creating turbulence;
permitting the oxygen-enriched liquid to exit the device; and
allowing the introduced gas to interact with microbiological material in at least one of the introduced waste water and/or a larger aqueous reservoir into which the oxygen enriched water is introduced.

10. The process of claim 9 wherein the gaseous oxygen is introduced at a rate sufficient to produce microbubbles, further comprising the steps of
introducing the processed water into a body of waste water, the waste water containing at least one of fats, oil, grease, biological matter or suspended solids; and
allowing the microbubbles to associate with the fats, grease or biological matter or suspended solids and sequester the same in a portion of the body of waste water.

11. The process of claim 9 wherein the introduced oxygen degrades phenolics in oil compounded wastewater which comprises carrying out one of a chemical and a microbiological reaction in a liquid comprising an oxygen-enriched liquid.

12. A fermentation process comprising the steps of:
fermenting a liquor comprising an oxygen-enriched liquid prepared by the process of claim 9.

13. An aerobic process which comprises carrying out one of a chemical and a microbiological reaction in a liquid comprising an oxygen-enriched liquid prepared by the process of claim 9.

14. A therapeutic process which comprises carrying out a therapeutic treatment of a body with an agent comprising an oxygen-enriched liquid prepared by the process of claim 9.

15. A process for bottling a potable beverage which comprises introducing a beverage comprising an oxygen-enriched liquid prepared using the apparatus of claim 1 into a container, and sealing said container.

16. A process of preparing a physiological saline solution which comprises the steps of:
proving oxygen enriched liquid prepared by the apparatus of claim 1; and dissolving a sodium concentrate into said oxygen enriched liquid.

17. A disinfection process which comprises subjecting microbial life to a liquid comprising an oxygen-enriched liquid prepared by the apparatus of claim 1.

18. A disinfection process which comprises enriching a liquid containing microbial life with oxygen the apparatus of claim 1.

19. The apparatus of claim 1, wherein the liquid is warm, with temperatures above 40 degrees Celsius.

20. An aerobic process in warm liquid which comprises carrying out one of a chemical and a microbiological reaction in a liquid comprising an oxygen-enriched prepared by the apparatus of claim 1.
